(19) United States
(12) Patent Application Publication
(10) Pub. No.: US 2006/0161153 A1
(43) Pub. Date: Jul. 20, 2006

(54) Pedicle screw systems and methods of assembling/installing the same

(75) Inventors: David T. Hawkes, Pleasant Grove, UT (US); Michael D. Ensign, Salt Lake City, UT (US); Thomas M. Sweeney II, Sarasota, FL (US); David R. Warnick, Spanish Fork, UT (US)

Correspondence Address: RADER, FISHMAN & GRAUER PLLC, 10653 SOUTH RIVER FRONT PARKWAY, SUITE 150, SOUTH JORDAN, UT, 84095, US

(73) Assignee: Alphaspine, Inc., Sarasota, FL (US)

(21) Appl. No.: 11/259,748

(22) Filed: Oct. 25, 2005

Related U.S. Application Data
(60) Provisional application No. 60/622,107, filed on Oct. 25, 2004.
(60) Provisional application No. 60/622,180, filed on Oct. 25, 2004.
(60) Provisional application No. 60/629,785, filed on Nov. 19, 2004.

(51) Int. Cl. A61F 2/30

(52) U.S. Cl. 606/61

(57) ABSTRACT

The pedicle screw system may be used for fixation of spinal segments and may be advantageous when minimally invasive surgery (MIS) techniques are employed. The pedicle screw system includes a tulip assembly comprising a tulip body, a inner member, an expansion member, and a cap assembly. Installation of the pedicle screw system into pedicles of the spine, for example, includes inserting the pedicle screw into a portion of the spine, coupling a partial assembly comprising the tulip body, inner member, and expansion member to the pedicle screw, placing a rod in the tulip assembly, and then coupling the cap assembly to the tulip body. Coupling the cap assembly to the tulip body includes initially locking the tulip assembly to the pedicle screw and then locking the rod in the tulip assembly.

### CROSS-REFERENCES TO RELATED APPLICATIONS

This application claims the benefit under 35 U.S.C. § 119(e) of U.S. Provisional Patent Application Nos. 60/622,107 filed Oct. 25, 2004; 60/622,180 filed Oct. 25, 2004; and 60/629,785 filed Nov. 19, 2004, where these provisional applications are incorporated herein by reference in their entireties.

### BACKGROUND OF THE INVENTION

1. Field of the Invention

The present invention relates generally to bone fixation devices, and in particular to a screw assembly for the internal fixation of vertebral bodies.

2. Description of the Related Art

Various devices for internal fixation of bone segments in the human or animal body are known in the art. One type of system is a pedicle screw system, which is sometimes used as an adjunct to spinal fusion surgery, and which provides a means of gripping a spinal segment. A conventional pedicle screw system comprises a pedicle screw and a rod-receiving device. The pedicle screw includes an externally threaded stem and a head portion. The rod-receiving device couples to the head portion of the pedicle screw and receives a rod (commonly referred to as a distraction rod). Two such systems are inserted into respective vertebrae and adjusted to distract and/or stabilize a spinal column, for instance during an operation to correct a herniated disk. The pedicle screw does not, by itself, fixate the spinal segment, but instead operates as an anchor point to receive the rod-receiving device, which in turn receives the rod. One goal of such a system is to substantially reduce and/or prevent relative motion between the spinal segments that are being fused.

Although conventional prior art pedicle screw systems exist, they lack features that enhance and/or benefit newer, minimally invasive surgery (MIS) techniques that are more commonly being used for spinal surgeries. It has been suggested that one possible advantage of an MIS approach is that it can decrease a patient's recovery time.

Conventional pedicle screw systems and even more recently designed pedicle screw systems have several drawbacks. Some of these pedicle screw systems are rather large and bulky, which may result in more tissue damage in and around the surgical site when the pedicle screw system is installed during surgery. The prior art pedicle screw systems have a rod-receiving device that is pre-operatively coupled or attached to the pedicle screw. In addition, some of the prior art pedicle screw systems include numerous components that must all be carefully assembled together. For example, one type of pedicle screw system that may require up to nine (9) different components is disclosed in U.S. Published Patent Application Nos. 2005/0203516 and 2005/0216003 to Biedermann et al.

One drawback that is common among many prior art pedicle screw systems is that a threaded component is used to lock down the rod in the rod-receiving device. Examples of these types of systems can be found in U.S. Published Patent Application Nos. 2005/0192571 to Abdelgany; 2005/0192573 to Abdelgany et al.; the Biedermann et al. applications; 2005/0187548 to Butler et al.; 2005/0203515 to Doherty et al.; and 2004/0172022 to Landry et al. Each of these pedicle screw systems have an externally threaded fastening element either directly or indirectly coupled to the vertically extending walls of the rod-receiving device (e.g., referred to as a bone fixator, a receiving part, a coupling construct, etc.).

One problem associated with the above-identified pedicle screw systems is that cross-threading may occur when the fastening element is installed. Cross-threading may cause the fastening element to jam and/or may result in an improper construct where some components may not be in the correct position. Due to the dynamic nature of spinal movement, a cross-threaded pedicle screw system may be more prone to post-operative failure.

Another problem with the above-identified pedicle screw systems is that the coupling between the fastening element and the rod-receiving device when subjected to dynamic, post-operative loading may result in the walls of the rod-receiving device splaying apart. In the above-identified pedicle screw systems, the walls of the rod-receiving device are unsupported. Post-operative tulip splaying, as it is commonly called, may result in the dislodgment of the fastening element and the rod. In short, the pedicle screw system may become post-operatively disassembled and no longer function according to its intended purpose.

Other prior art pedicle screw systems have attempted to address some of the aforementioned drawbacks. For example, U.S. Pat. Nos. 5,609,593, 5,647,873, 5,667,508, 5,669,911, and 5,690,630, all to Errico et al., disclose a threaded, outer cap that extends over and couples to the walls of the rod-receiving device. However, the risk and/or potential for cross-threading is still present when the threaded, outer cap is coupled with the rod-receiving device.

Other pedicle screw systems such as U.S. Pat. Nos. 5,882,350 to Ralph et al.; 6,132,432 to Richelsoph; 4,950,269 to Gaines, Jr.; 6,626,908 to Cooper et al.; 6,402,752 to Schäffler-Wachter et al.; and 6,843,791 to Serhan may address some of the aforementioned drawbacks, but each of these pedicle screw systems are pre-operatively assembled, which makes these systems more difficult to install and maneuver in a spinal operation where MIS techniques are used.

### BRIEF SUMMARY OF THE INVENTION

The embodiments described herein are generally related to a bone fixation assembly or pedicle screw system for the internal fixation of vertebral bodies. The pedicle screw system may be used to fix, correct, stabilize, and/or reinforce spinal segments, among other things and may be particularly advantageous when minimally invasive surgery (MIS) techniques are employed. The pedicle screw system includes a tulip assembly comprising a tulip body, a inner member, an expansion member, and a cap assembly. Installation of the pedicle screw system into pedicles of the spine, for example, includes inserting the pedicle screw into a portion of the spine, coupling a partial assembly comprising the tulip body, inner member, and expansion member to the pedicle screw, placing a rod in the inner member, and then coupling the cap assembly to the tulip body. The partial assembly may be locked onto the pedicle screw before or after placement of the rod. In addition, the cap assembly operates to further lock the tulip assembly to the pedicle screw and to capture and lock the rod with the tulip assembly.

In one aspect, a tulip assembly is configured to receive a rod and is coupleable to a pedicle screw having a head portion. The tulip assembly includes a first member having an upper portion and a lower portion. The upper portion includes a cap-engagement portion. The lower portion includes a bore with a first surface. A ring is expandable over the head portion of the pedicle screw and the ring includes an outer surface seated in the lower portion of the first member with at least a portion of the outer surface in contact with a portion of the first surface of the first member. A inner member is positioned within the first member and includes a contact portion to urge the ring into tight contact with the head portion of the pedicle screw and with the first surface of the first member. A cap assembly includes an inner cap and an outer cap. The inner cap includes a first portion and a second portion, where the first portion is rotatable with the inner cap to contact the inner member, and where the second portion is rotatable with the inner cap to contact the rod. And, the outer cap is engageable with the cap-engagement portion of the first member.

In another aspect, a pedicle screw system includes a pedicle screw having a threaded portion and a head portion; and a tulip assembly having a first member, a ring, a inner member, and a cap assembly. The first member of the tulip assembly includes an upper portion and a lower portion. The upper portion includes a cap-engagement portion. The lower portion includes a bore with a first surface. The ring of the tulip assembly is expandable over the head portion of the pedicle screw. The ring includes an outer surface seated in the lower portion of the first member with at least a portion of the outer surface in contact with a portion of the first surface of the first member. The inner member of the tulip assembly is positioned within the first member and includes a contact portion to urge the ring into tight contact with the head portion of the pedicle screw and with the first surface of the first member. The cap assembly of the tulip assembly includes an inner cap and an outer cap. The inner cap includes a first portion and a second portion, where the first portion is rotatable with the inner cap to contact the inner member, and where the second portion is rotatable with the inner cap to contact the rod. And, the outer cap is engageable with the cap-engagement portion of the first member.

In yet another aspect, a method of locking a tulip assembly to a pedicle screw includes inserting the pedicle screw into bone, the pedicle screw having a head portion that includes a maximum diameter section; expanding a ring that is positioned in a first member over and past the maximum diameter section of the pedicle screw; and rotating an inner cap of a cap assembly a first amount to apply a force onto a inner member that is in contact with and positioned above the ring within the first member, wherein rotating the inner cap urges the ring downward along an inner surface of a bore of the first member to frictionally lock the first member to the head portion of the pedicle screw.

In still yet another embodiment, a method of locking a tulip assembly to a pedicle screw and locking a rod in the tulip assembly includes inserting the pedicle screw into bone, the pedicle screw having a head portion that includes a maximum diameter section; expanding at least a portion of a ring that is positioned in a first member over and past the maximum diameter section of the pedicle screw; rotating an inner cap of a cap assembly a first amount to apply a force onto a inner member that is in contact with and positioned above the ring within the first member, wherein rotating the inner cap urges the ring downward to frictionally lock the first member to the head portion of the pedicle screw; and rotating the inner cap of the cap assembly a second amount to lock at least a portion of the rod within a region between the inner cap and the inner member.

### BRIEF DESCRIPTION OF THE SEVERAL VIEWS OF THE DRAWING(S)

In the drawings, identical reference numbers identify similar elements or acts. The sizes and relative positions of elements in the drawings are not necessarily drawn to scale. For example, the shapes of various elements and angles are not drawn to scale, and some of these elements are arbitrarily enlarged and positioned to improve drawing legibility. Further, the particular shapes of the elements as drawn, are not intended to convey any information regarding the actual shape of the particular elements, and have been solely selected for ease of recognition in the drawings. In the drawings, identical reference numbers identify similar elements, assemblies, acts, and/or steps.

FIG. 1 is an isometric view of a pedicle screw system and a distraction rod, according to one illustrated embodiment.

FIG. 2 is side elevational view of a pedicle screw having a variable minor diameter, according to one illustrated embodiment.

FIG. 3 is an isometric, exploded view showing a pedicle screw and various components of a tulip assembly of the pedicle screw system of FIG. 1.

FIG. 4 is a cross-sectional view of a split ring received in a tulip body, both of which are components of the tulip assembly of FIG. 3.

FIG. 5 is an isometric view of a inner member, which is a component of the tulip assembly of FIG. 3.

FIG. 6 is an isometric view of an inner cap of a cap assembly, which is also a component of the tulip assembly of FIG. 3.

FIG. 7 is an isometric view of an outer cap of a cap assembly, which is also a component of the tulip assembly of FIG. 3.

FIG. 8 is a flow diagram describing a method of installing a pedicle screw assembly, according to one embodiment.

### DETAILED DESCRIPTION OF THE INVENTION

In one embodiment, pedicle screw systems may be fixed in the spine, for example to perform spinal fixation and/or corrective surgeries, which surgeries may be performed via minimally invasive surgery (MIS) techniques. The systems are inserted into the pedicles of the spine and then interconnected with rods to manipulate (e.g., correct the curvature, compress or expand, and/or structurally augment) at least portions of the spine. Using the MIS approach to spinal fixation and/or correction surgery has been shown to decrease a patient's recovery time and reduce the risks of follow-up surgeries.

The ability to efficiently perform spinal fixation and/or correction surgeries using MIS techniques is enhanced by the use of pedicle screw systems provided in accordance with the present invention, which systems provide many advantages over conventional systems. For example, a pedicle screw system in accordance with one embodiment provides the advantage that the pedicle screw may be inserted into the bone without being pre-operatively coupled with the rod-coupling assembly (hereinafter referred to as a tulip assembly). This is advantageous because the surgeon often needs to do other inter-body work after inserting the pedicle screw, but before attaching the larger and bulkier tulip assembly. Such an advantageous pedicle screw system may be even more crucial when using MIS techniques because the inter-body spatial boundaries in which the surgeon must work may be quite limited.

In addition, pedicle screw systems in accordance with the present invention advantageously allow a user to initially fix (e.g., lock) the tulip assembly to the pedicle screw at a desired angle before inserting and/or capturing the rod. Initially locking the tulip assembly to the pedicle screw means that at least one of the components of the tulip assembly is manipulated to grip and/or clamp onto the pedicle screw to reduce, if not prevent any translational and/or rotational movement of the tulip assembly relative to the pedicle screw. The ability to initially lock the tulip assembly to the pedicle screw may facilitate the surgeon in performing compression and/or distraction of various spinal and/or bone sections.

The term “distraction,” when used in a medical sense, generally relates to joint surfaces and suggests that the joint surfaces move perpendicular to one another. However when “traction” and/or “distraction” is performed, for example on spinal sections, the spinal sections may move relative to one another through a combination of distraction and gliding.

In addition to accommodating the new MIS approach to spinal correction and/or fusion, at least one pedicle screw system described herein may include features to prevent, or at least reduce, the problems of cross-threading and/or post-operative tulip splaying, which is when the amount of stress/strain in rod forces open the tulip assembly and eventually leads to disassembly and/or failure of the pedicle screw system.

Further, another possible advantage of the pedicle screw system of the present invention is that the rod is locked in contact with the tulip body, but does not contact (i.e., load) the head portion of the pedicle screw.

Pedicle Screw System

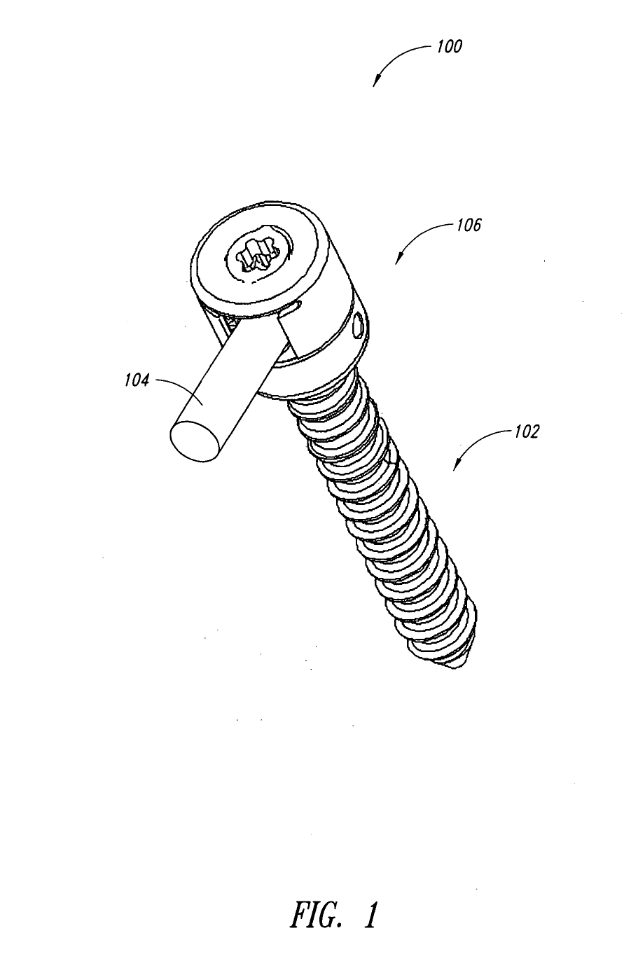

FIG. 1 generally shows a pedicle screw system 100 comprising a pedicle screw 102 and a coupling assembly 106, hereinafter referred to as a tulip assembly 106, where a rod 104 is received in the tulip assembly 106. The placement and/or number of pedicle screw systems 100 for a patient may be pre-operatively determined based on a pre-operative examination of the patient's spinal system using non-invasive imaging techniques known in the art, such as x-ray imaging, magnetic resonance imaging (MRI), and/or fluoroscopy imaging, for example. The tulip assembly 106 is intra-operatively (i.e., during surgery) coupleable to the pedicle screw 102 and maneuverable to achieve a desired placement, orientation, and/or angular position of the tulip assembly 106 relative to the pedicle screw 102. Once the tulip assembly 106 is at the desired position relative to the pedicle screw 102, the tulip assembly 106 can be fixed or locked onto the pedicle screw 102. In one embodiment, the tulip assembly 106 is fixed onto the pedicle screw 102 before the rod 104 is fixed or locked into the tulip assembly 102. In another embodiment, the tulip assembly 106 is fixed onto the pedicle screw 102 contemporaneously as the rod 104 is fixed or locked into the tulip assembly 102.

It is understood that the relative, angular position of a first tulip assembly 106 to a first pedicle screw 102 may be different from other pedicle screw systems 100 located elsewhere on a patient's spine. In general, the relative position of the tulip assembly 106 to the pedicle screw 102 allows the surgeon to selectively and independently orient and manipulate the tulip assemblies 106 to achieve the goals commensurate with the surgical procedure, which may involve compressing, expanding, distracting, rotating, and/or otherwise correcting an alignment of at least a portion of a patient's spine.

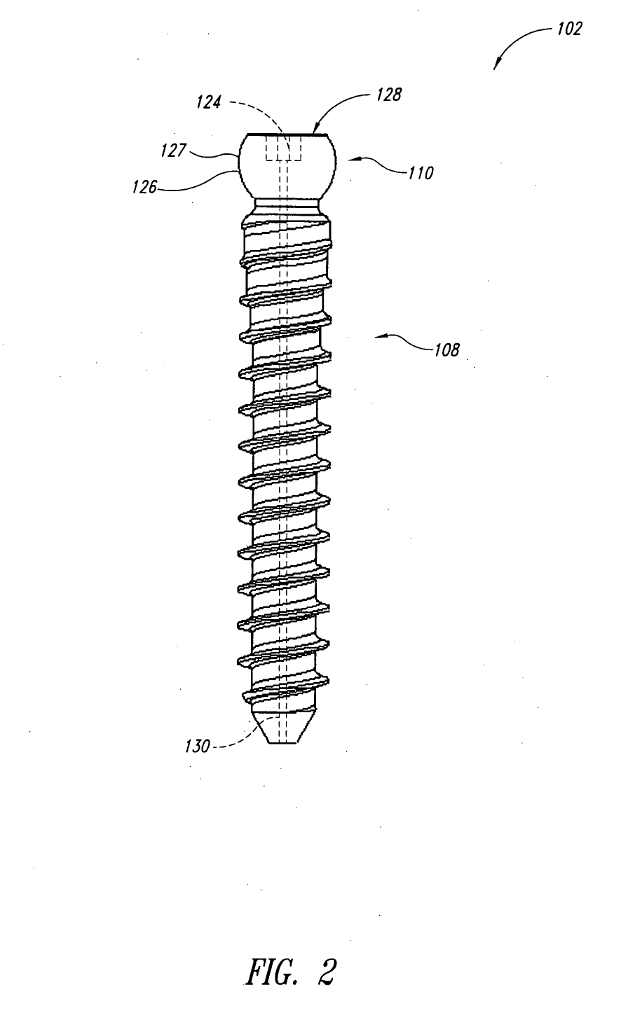

FIG. 2 shows the pedicle screw 102 having an elongated, threaded portion 108 and a head portion 110.

Although pedicle screws 102 are generally known in the art, the head portions 110 may be of varying configurations depending on what type of tulip assembly 106 is to be coupled to the pedicle screw 102. The head portion 110 of the pedicle screw 102 includes a driving feature 124 and a maximum diameter portion 126. The driving feature 124 permits the pedicle screw 102 to be inserted into a pedicle bone and/or other bone. The pedicle bone is a part of a vertebra that connects the lamina with a vertebral body. The driving feature 124 can be used to adjust the pedicle screw 102 even after the tulip assembly 106 is coupled to the pedicle screw 102. In the illustrated embodiment, the head portion 110 of the pedicle screw 102 is coupled to the threaded portion 108 and includes a generally spherical surface 127 with a truncated or flat top surface 128.

In one embodiment, the pedicle screw 102 is cannulated, which means a channel 130 (shown in dashed lines and extending axially through the pedicle screw 102) extends through the entire length of the pedicle screw 102. The channel 130 allows the pedicle screw 102 to be maneuvered over and receive a Kirschner wire, commonly referred to as a K-wire. The K-wire is typically pre-positioned using imaging techniques, for example, fluoroscopy imaging.

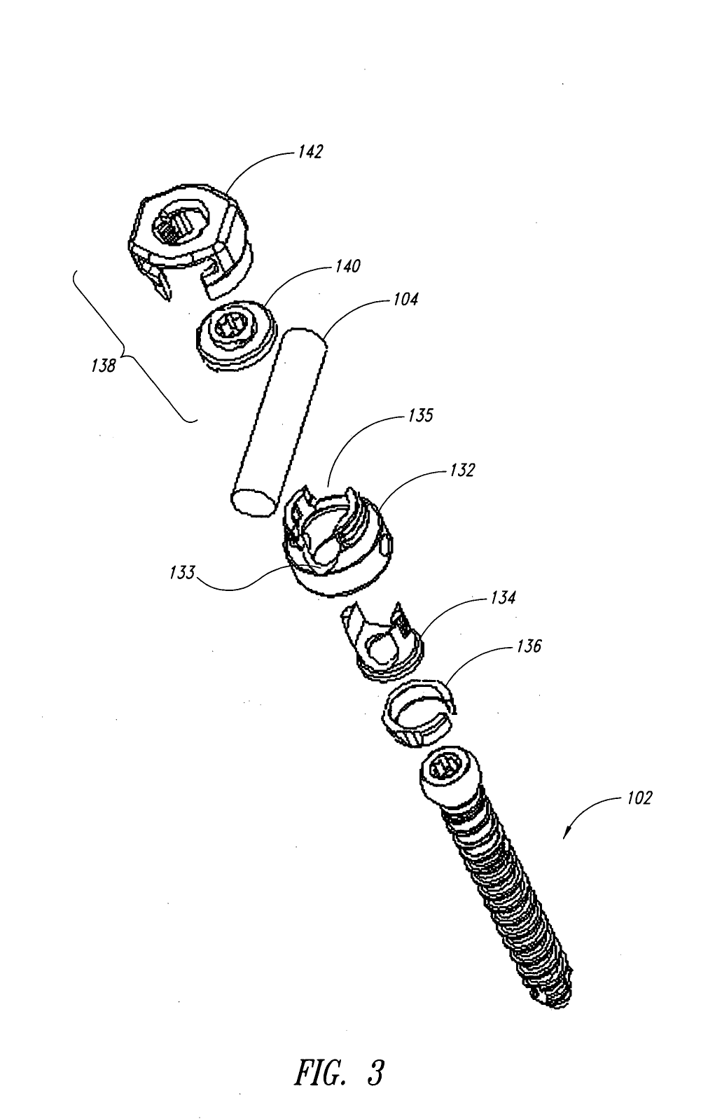

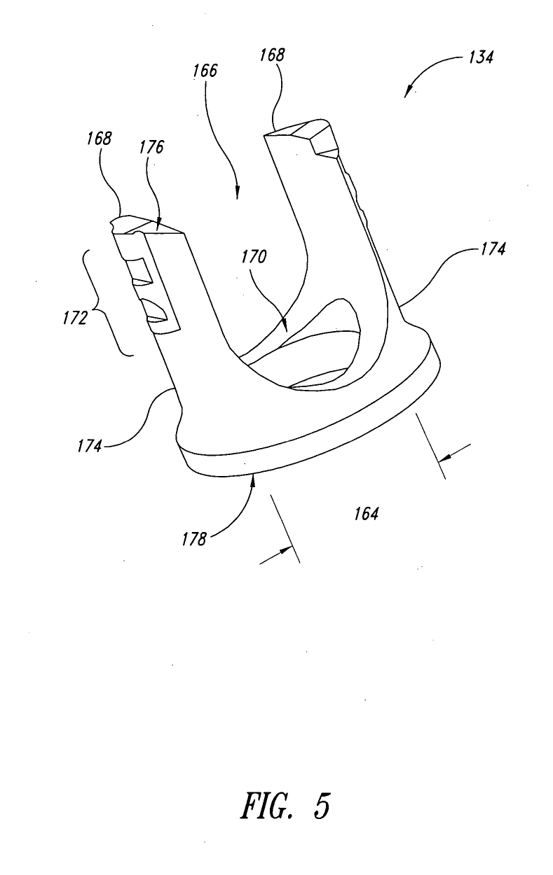

FIG. 3 shows the tulip assembly 106 that includes a first member or tulip body 132, an inner member or inner member 134, an expansion/contraction member or split ring 136, and a cap assembly 138, according to one illustrated embodiment. For the purposes of the description herein, the rod 104 and the pedicle screw 102 are not considered to be part of the tulip assembly 106. In one embodiment, the tulip body 132, the inner member 134, and the split ring 136 are pre-operatively assembled before being placed onto the head portion 110 of the pedicle screw 102. Both the inner member 134 and the split ring 136 are received into the tulip body 132 through a bottom opening. In one embodiment, the tulip body 132 includes a surface 133 to receive and substantially support at least a portion of the rod 104, where the portion of the rod 104 has little or no contact with a surface 170 (FIG. 5) of the inner member 134. Alternatively, the surface 133 of the tulip body cooperates with the surface 170 (FIG. 5) to receive and support the portion of the rod 104.

The cap assembly 138 comprises an inner cap 140 and an outer cap 142. In one embodiment, the inner cap 140 and the outer cap 142 may also be pre-operatively coupled together before the cap assembly 138 is coupled to the tulip body 132.

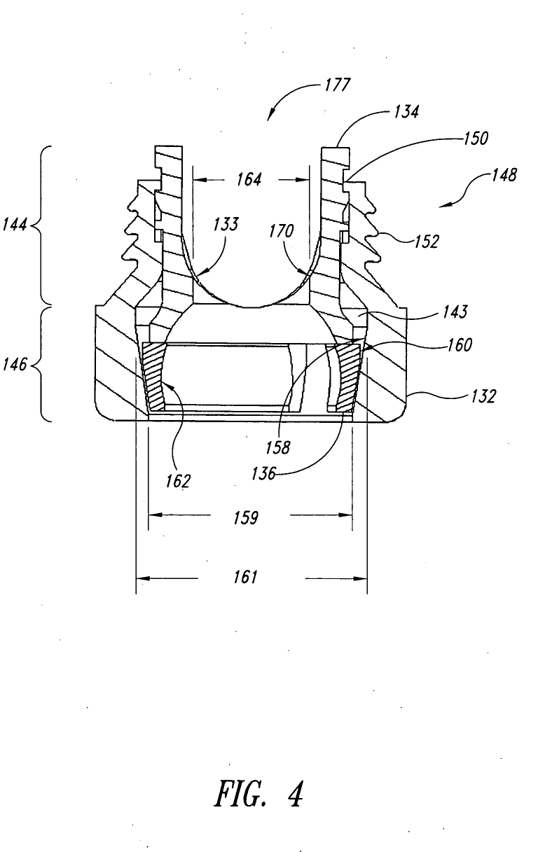

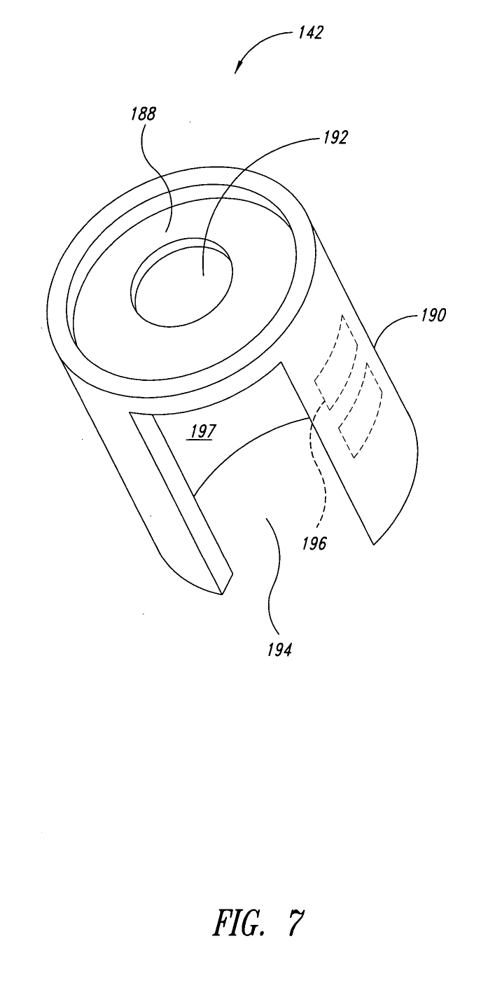

FIG. 4 shows the split ring 136 in the tulip body 132. For purposes of clarity, the pedicle screw 102 and the inner member 134 are not shown. The tulip body 132 includes a bore 143, an upper portion 144, and a lower portion 146. The upper portion 144 includes an outer engagement portion 148 and a lip 150. In the illustrated embodiment, the outer engagement portion 148 is a plurality of ridges/detents 152 to receive complementary ridges 196 (FIG. 7) provided along an inner surface 197 (FIG. 7) of the outer cap 142. Similarly, the lip 150 extends radially inward to receive and longitudinally retain the inner member 134 via engagement between the lip 150 of the tulip body 132 and the engagement portion 172 of the inner member 134.

An inner surface 158 of the bore 143 in the lower portion 146 of the tulip body 132 is sized to allow the split ring 136 to float and/or translate upwards toward the upper portion 144 where the split ring 136 can expand to receive the head portion 110 of the pedicle screw 102. In one embodiment, the bore 143 is curved or tapered from a first diameter region 159 to a second, larger diameter region 161, where the second, larger diameter region 161 is located closer and/or proximate to the upper portion 144 of the tulip body 132. The split ring 136 includes an outer surface 160 and an inner surface 162. The outer surface 160 of the split ring 136 frictionally contacts the inner surface 158 of the bore 143. The inner surface 162 frictionally engages the head portion 110 of the pedicle screw 102, as will be described in more detail below.

FIG. 5 shows the inner member 134 having a minimum inner diameter 164 (FIG. 4), a U-shaped channel 166 formed by extending arms 168, the surface 170, an engagement portion 172 located on an exterior surface 174 of at least one of the arms 168, a top surface 176, and a bottom surface 178. The minimum inner diameter 164 is sized to be smaller than the maximum diameter portion 126 (FIG. 2) of the head portion 110 of the pedicle screw 102. The engagement portion 172 of the inner member 134 comprises a plurality of ridges/detents to engage with the plurality of ridges/detents 152 of the tulip body 132. The top surface 176 is contoured to cooperate with the inner cap 140 of the cap assembly 138, as will be described below. The bottom surface 178 operates to engage the split ring 136 and force the split ring 136 down in the bore 143 of the tulip body 132, which results in contraction of the split ring 136 around the head portion 110 of the pedicle screw 102. It is understood that the forced contraction of the split ring 136 along with the radial constraint provided by the inner surface 158 of the tulip body 132 generates sufficient radial pressure on the head portion 110 of the pedicle screw 102 to lock the tulip body 132 onto the pedicle screw 102.

The U-shaped channel 166 is alignable with a similar channel 135 (FIG. 3) of the tulip body 132. When the U-shaped channel 166 and the channel 135 are aligned, and when the cap assembly 138 is placed on the tulip body 132, a region, referred to as a rod-receiving region 177 (FIG. 4), includes boundaries formed substantially by the surface 133 of the tulip body 132, the surface 170 of the inner member 134, and the inner cap 140. As will be described in more detail below, the portion of the rod 104 may initially be in contact with the surface 170 of the inner member 134, but as the inner member 134 is urged downward toward the pedicle screw 102, the portion of the rod 104 may lose contact with the surface 170 of the inner member 134 and end up being in contact with only the surface 133 of the tulip body 132.

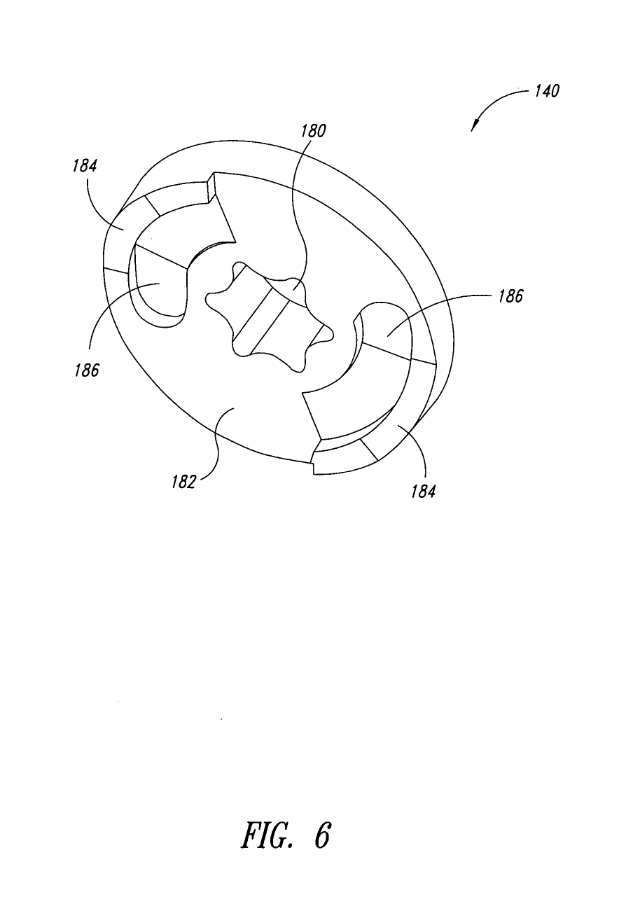

FIG. 6 shows the inner cap 140 of the cap assembly 138. The inner cap 140 includes an opening 180 and a bottom portion 182 having a first ramp 184 and a second ramp 186. The first ramp 184 is radially further from the opening 180 than the second ramp 186. In the illustrated embodiment, the first ramp 184 protrudes from the bottom portion 182 while the second ramp 186 recesses into the bottom portion 182. Alternatively, both ramps 184, 186 could be protruding, both recessed, or the first ramp 184 could be recessed and the second ramp 186 protruding. It is understand that the aspects such as slope, length, recessed or protruding ramps, etc. may be selected and/or even optimized for manufacturing purposes.

FIG. 7 shows the outer cap 142 having a top portion 188 coupled to an axially and/or longitudinally extending wall 190. The top portion 188 includes a first opening 192 to allow access to the pedicle screw 102. The wall 190 includes an open section 194 to allow the outer cap 142 to be placed over the rod 104 after the rod 104 has been placed in the inner member 134 of the tulip assembly 106 (FIG. 1). The open section 194 is shown as having a square or rectangular profile, however it is understood that the opening section 194 may comprise other profiles, for example U-shaped. The outer cap 142 further includes an engagement portion 196 on the interior of the outer cap 142 to couple to the engagement portion 172 (FIG. 5) of the inner member 134.

Pedicle Screw System Installation

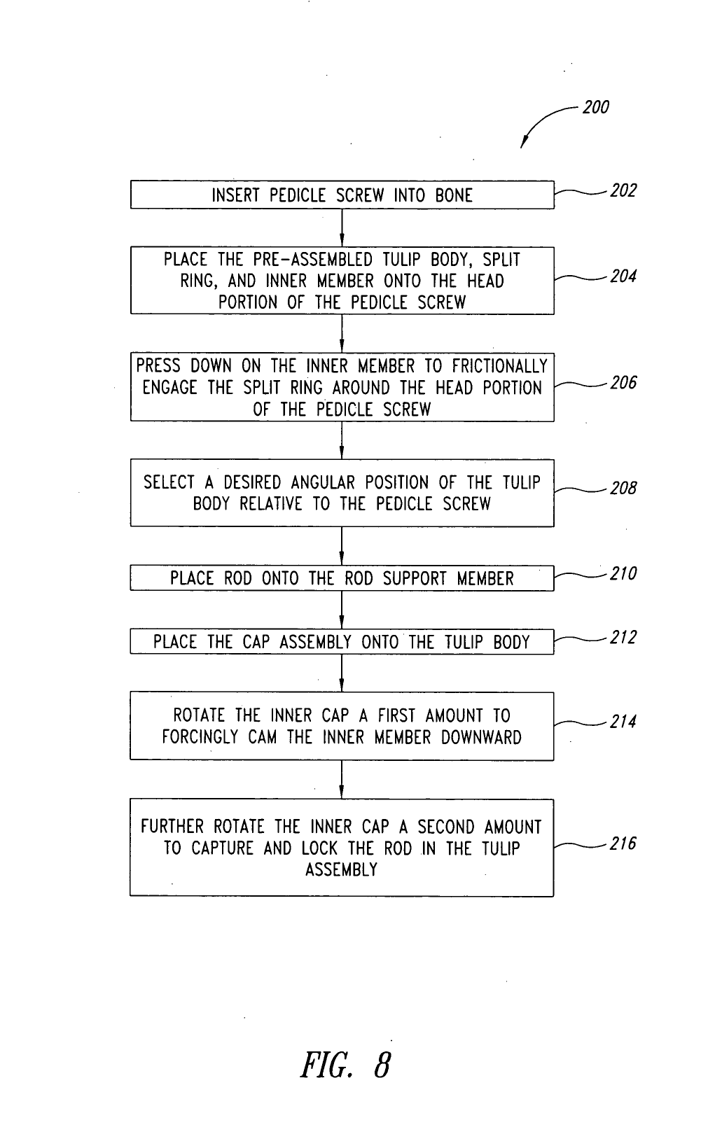

FIG. 8 is a flow diagram showing a method 200 of installing the pedicle screw system 100 into bone. Reference to FIG. 1-7 may be of assistance when following the various steps of the method 200. After a surgical site is prepped and ready, the pedicle screw 102 is driven and/or otherwise inserted into the patient's bone, step 202. The pre-assembled tulip body 132, inner member 134, and split ring 136 (hereinafter referred to as the partially assembled tulip) are placed onto the head portion 110 of the pedicle screw 102, step 204.

Pre-assembling the tulip body 132, inner member 134, and the split ring 136 comprises inserting the inner member 134 and split ring 136 up through the bottom of the tulip body 132, according to one embodiment. At this point, the split ring 136 is permitted to float within the bore 143 of the lower portion 146 of the tulip body 132. As the partially assembled tulip is pressed onto the pedicle screw 102 the split ring 136 moves upward along the inner surface 160 (FIG. 4) of the tulip body 132, which allows the split ring 136 to expand over the head portion 110 of the pedicle screw 102.

The surgeon or other member of the surgical staff then presses down on the inner member 134, step 206. This downward pressure on the inner member 134 causes the bottom surface 178 thereof to force the split ring 136 down onto the head portion 110 of the pedicle screw 102 while the inner surface 160 of the tulip body 132 operates to radially constrain and reduce the diametrical size of the split ring 132. Alternatively stated, the inner member 134 and the tulip body 132 cooperate to force the split ring 136 into frictional engagement with the head portion 110 of the pedicle screw 102. The curvature of the inner surface 162 of the split ring 136 allows the split ring 136 to hug or closely receive the head portion of the pedicle screw 102. At this point, the partially assembled tulip is rotatably coupled (i.e., free to rotate poly-axially) to the head portion 110 of the pedicle screw 102, yet cannot be pulled off the pedicle screw 102 unless an appropriate tool is used to manipulate the split ring 136 back up into the wider portion of the bore 134 of the tulip body.

The partially assembled tulip may be rotationally oriented with respect to the pedicle screw 102, step 208. This advantageously permits the surgeon to intra-operatively select and achieve an angular position of the tulip body 132 relative to the pedicle screw 102. The rod 104 is then placed into the rod-receiving region 177 and may, at least initially, be in contact with the surface 170 of the inner member 134, step 210.

The cap assembly 138 is placed onto the tulip body 132, step 212. The engagement portion 196 of the outer cap 142 engages the engagement portion 148 of the tulip body 132. In one embodiment, the engagement portions are complementary ridges/detents 152, 196 of the tulip body 132 and the outer cap 142, respectively. This allows the outer cap 142 to successively engage (e.g., snap-on) onto the tulip body 132. As the outer cap 142 is pushed or snapped down onto the tulip body 132, the engagement portion 172 of the inner member 134 to force the inner cap 140 into contact with the upper surface 168 of the inner member 134. The outer cap 142 may, if desired, be forced and/or snapped as far down as possible onto the tulip body 132. It is understood and appreciated that the engagement portions 172, 196 are complementary to one another. In one embodiment, ridges/detents of the tulip body 132 operate to engage with the complementary ridges/detents of the outer cap 142. In addition, other equivalent means can be used to couple the cap assembly 138 to the tulip body 132.

At this point, the rod 104 is captured in the tulip assembly 106 and the second ramp 184 of the inner cap 140 is in contact with the upper surface 168 of the inner member 134. The inner cap 140 is rotated a first amount such that the second ramp 184 forcingly cams the inner member 134 downward, step 214. This camming action of the second ramp 184 of the inner cap 140 with the upper surface 168 of the inner member 134 urges the inner member 134 into tighter contact with the split ring 136, which in turn forces the split ring 136 further down the surface 158 of the tulip body 132 and into tighter contact with the head portion 110 of the pedicle screw 102, thereby further securing the tulip assembly 106 to the pedicle screw 102. In addition, the inner member 134 is longitudinally retained by the tulip body 132 via engagement of the lip 150 of the tulip body 132 and the engagement portion 172 of the inner member (FIG. 4). Additionally or alternatively, the portion of the rod 104 may lose contact with the surface 170 of the inner member 134; yet remain in contact with the surface 133 of the tulip body 132.

The inner cap 140 is then rotated a second amount, which brings the second ramp 184 out of contact with the upper surface 168 of the inner member 134 and brings the first ramp 184 into contact with the rod 104, step 216. In one embodiment, the profile of the first ramp 186 forcingly cams the rod 104 into tight contact with the surface 133 of the tulip body 132 and thus locks the rod 104 in the tulip assembly 106. In another embodiment, the first ramp 186 forcingly cams the rod 104 into tight contact with the surface 133 of the tulip body 132 and/or with the surface 170 of the inner member 134 to lock the rod 104 in the tulip assembly 106. It is understand that whether or not the portion of the rod 104 contacts the surface 170 of the inner member 134 depends on how far downward the inner member 134 is forced toward the pedicle screw 102 when the cap assembly 138 is installed. The tulip assembly 106 and rod 104 are now locked or fixed relative to the pedicle screw 102, which completes the installation for at least one embodiment of a pedicle screw system.

The above description of illustrated embodiments, including what is described in the Abstract, is not intended to be exhaustive or to limit the invention to the precise forms disclosed. Although specific embodiments of and examples are described herein for illustrative purposes, various equivalent modifications can be made without departing from the spirit and scope of the invention, as will be recognized by those skilled in the relevant art. The teachings provided herein of the invention can be applied to various screws, not necessarily the exemplary pedicle screws generally described above.

The various embodiments described above can be combined to provide further embodiments. All of the U.S. patents, U.S. patent application publications, U.S. patent applications, foreign patents, foreign patent applications and non-patent publications referred to in this specification and/or listed in the Application Data Sheet, to include U.S. Provisional Patent Application Nos. 60/622,107 filed Oct. 25, 2004; 60/622,180 filed Oct. 25, 2004; and 60/629,785 filed Nov. 19, 2004, are incorporated herein by reference, in their entirety. Aspects of the invention can be modified, if necessary, to employ screws, materials and concepts of the various patents, applications and publications to provide yet further embodiments of the invention.

These and other changes can be made to the invention in light of the above-detailed description. In general, in the following claims, the terms used should not be construed to limit the invention to the specific embodiments disclosed in the specification and the claims, but should be construed to include all spine anchoring devices that operated in accordance with the claims. Accordingly, the invention is not limited by the disclosure, but instead its scope is to be determined entirely by the following claims.

1. A tulip assembly configured to receive a rod and coupleable to a pedicle screw having a head portion, the tulip assembly comprising:
a first member having an upper portion and a lower portion, the upper portion having a cap-engagement portion, the lower portion having a bore with a first surface;
a ring expandable over the head portion of the pedicle screw, the ring having an outer surface seated in the lower portion of the first member with at least a portion of the outer surface in contact with a portion of the first surface of the first member;
a inner member positioned within the first member, the inner member having a contact portion to urge the ring into tight contact with the head portion of the pedicle screw and with the first surface of the first member; and
a cap assembly comprising an inner cap and an outer cap, the inner cap having a first portion and a second portion, the first portion rotatable with the inner cap to contact the inner member, the second portion rotatable with the inner cap to contact the rod, the outer cap engageable with the cap-engagement portion of the first member.

2. The tulip assembly of claim 1 wherein the ring is elastically, diametrically expandable and contractible.

3. The tulip assembly of claim 1 wherein the cap-engagement portion of the first member includes a protuberance extending from the upper portion of the first member.

4. The tulip assembly of claim 3 wherein the outer cap includes a protuberance to engage with the protuberance of the first member.

5. The tulip assembly of claim 1 wherein the bore of the first member is configured to permit longitudinal movement of the ring adjacent to the first surface of the bore.

6. A pedicle screw system comprising:
a pedicle screw having a threaded portion and a head portion; and
a tulip assembly comprising a first member, a ring, a inner member, and a cap assembly, the first member having an upper portion and a lower portion, the upper portion having a cap-engagement portion, the lower portion having a bore with a first surface,
the ring expandable over the head portion of the pedicle screw, the ring having an outer surface seated in the lower portion of the first member with at least a portion of the outer surface in contact with a portion of the first surface of the first member;
the inner member positioned within the first member, the inner member having a contact portion to urge the ring into tight contact with the head portion of the pedicle screw and with the first surface of the first member; and
the cap assembly comprising an inner cap and an outer cap, the inner cap having a first portion and a second portion, the first portion rotatable with the inner cap to contact the inner member, the second portion rotatable with the inner cap to contact the rod, the outer cap engageable with the cap-engagement portion of the first member.

7. The pedicle screw system of claim 6 wherein the ring is elastically, diametrically expandable and contractible.

8. The pedicle screw system of claim 6 wherein the cap-engagement portion of the first member includes a protuberance extending from the upper portion of the first member.

9. The pedicle screw system of claim 8 wherein the outer cap includes a protuberance to engage with the protuberance of the first member.

10. The pedicle screw system of claim 6 wherein the bore of the first member is configured to permit longitudinal movement of the ring adjacent to the first surface of the bore.

11. A method of locking a tulip assembly to a pedicle screw, the method comprising:
inserting the pedicle screw into bone, the pedicle screw having a head portion that includes a maximum diameter section;
expanding a ring that is positioned in a first member over and past the maximum diameter section of the pedicle screw; and
rotating an inner cap of a cap assembly a first amount to apply a force onto a inner member that is in contact with and positioned above the ring within the first member, wherein rotating the inner cap urges the ring downward along an inner surface of a bore of the first member to frictionally lock the first member to the head portion of the pedicle screw.

12. The method of claim 11 wherein inserting the pedicle screw into bone includes screwing the pedicle screw into bone.

13. The method of claim 11 wherein expanding the ring includes moving the ring upward in the bore of the first member, wherein the bore includes a variable diameter.

14. The method of claim 11 wherein rotating the inner cap of the cap assembly by the first amount to apply the force onto the inner member includes contacting the inner member with a first ramp formed in the inner cap.

15. A method of locking a tulip assembly to a pedicle screw and locking a rod in the tulip assembly, the method comprising:
inserting the pedicle screw into bone, the pedicle screw having a head portion that includes a maximum diameter section;
expanding at least a portion of a ring that is positioned in a first member over and past the maximum diameter section of the pedicle screw;
rotating an inner cap of a cap assembly a first amount to apply a force onto a inner member that is in contact with and positioned above the ring within the first member, wherein rotating the inner cap urges the ring downward to frictionally lock the first member to the head portion of the pedicle screw; and
rotating the inner cap of the cap assembly a second amount to lock at least a portion of the rod within the tulip assembly.

16. The method of claim 15 wherein inserting the pedicle screw into bone includes screwing the pedicle screw into bone.

17. The method of claim 15 wherein expanding the ring includes moving the ring to upward in the bore of the first member, wherein the bore includes a variable diameter.

18. The method of claim 15 wherein rotating the inner cap of the cap assembly by the first amount to apply the force onto the inner member includes contacting the inner member with a first ramp formed in the inner cap.

19. The method of claim 15 wherein rotating the inner cap of the cap assembly the second amount includes contacting the rod with a second ramp formed in the inner cap.

20. A tulip assembly configured to receive a rod and coupleable to a pedicle screw having a head portion, the tulip assembly comprising:
a first member having an upper portion and a lower portion, the upper portion having a cap-engagement portion, the lower portion having a bore with a first surface;
a ring expandable over the head portion of the pedicle screw, the ring having an outer surface seated in the lower portion of the first member with at least a portion of the outer surface in contact with a portion of the first surface of the first member;
a inner member positioned within the first member, the inner member having a contact portion to urge the ring into tight contact with the head portion of the pedicle screw and with the first surface of the first member; and
a cap assembly comprising an inner cap and an outer cap, the inner cap having a first portion and a second portion, the first portion rotatable with the inner cap to contact the inner member, the second portion rotatable with the inner cap to contact the rod, the outer cap engageable with the cap-engagement portion of the first member,
wherein the rod contacts the tulip body and the inner cap without contacting the head portion of the pedicle screw.
